(19) United States
(12) Patent Application Publication
(10) Pub. No.: US 2012/0100590 A1
(43) Pub. Date: Apr. 26, 2012

(54) MICROBIALLY-ASSISTED WATER ELECTROLYSIS FOR IMPROVING BIOMETHANE PRODUCTION

(76) Inventors: Boris Tartakovsky, Cote Saint Luc (CA); Serge R. Guiot, Montreal (CA)

(21) Appl. No.: 13/376,867

(22) PCT Filed: Jun. 22, 2010
(86) PCT No.: PCT/CA2010/000966

§ 371 Date: Dec. 8, 2011

Related U.S. Application Data
(60) Provisional application No. 61/213,694, filed on Jul. 2, 2009.

(51) Int. Cl. C12P 5/02

(52) U.S. Cl. 435/167

(57) ABSTRACT

A method of producing in a bioreactor a biogas rich in methane involves electrolyzing water in an aqueous medium at a voltage in a range of from 1.8 V to 12 V in the presence of electrochemically active anaerobic microorganisms that biocatalyze production of hydrogen gas, and, contacting a species of hydrogenotrophic methanogenic microorganisms with the hydrogen gas and carbon dioxide to produce methane. Volumetric power consumption is in a range of from 0.03 Wh/L_{R} to 0.3 Wh/L_{R}. Current density is 0.01 A/cm_{E}² or lower. The voltage is sufficient to electrolyze water without destroying microbial growth. Such a method results in improved electrolysis efficiency while avoiding the use of noble metal catalysts. Further, a combination of water electrolysis with anaerobic degradation of organic matter results in increased biogas quality and in increased biogas quantity and yield. Oxidation of hydrogen sulfide contributes to the increased quality, while an increase in the rate of organic matter hydrolysis and an increase in the production of methane from hydrogen contributes to the increased quantity and yield.

### CROSS-REFERENCE TO RELATED APPLICATIONS

This application claims the benefit of U.S. Provisional Patent Application Ser. No. 61/213,694 filed Jul. 2, 2009, the entire contents of which is herein incorporated by reference.

### FIELD OF THE INVENTION

The present invention relates to methane production, in particular to a method and apparatus involving water electrolysis in the presence of microorganisms to produce hydrogen for conversion to methane in an anaerobic reactor.

### BACKGROUND OF THE INVENTION

Anaerobic digestion (AD) combines solid organic waste or wastewater biotreatment with methane production and can be used to treat a broad range of organic compounds. There are several commercial versions of this process for wet digestion, that are designed to treat wastewaters with a high COD concentration (more than 1.5-2 g-COD/L), or to reduce organic solid content of organic solid suspensions or slurries (up to 15% total solid content). Recent demand for renewable energy sources have boosted AD research and applications, nevertheless several restrictions characteristic of the AD process limit its application for energy recovery from organic wastes. The main restrictions include relatively high influent concentrations of organic matter required for the successful operation of anaerobic reactors, slow anaerobic hydrolysis of complex organic materials, high concentrations of carbon dioxide (up to 50%) and the presence of hydrogen sulfide in the biogas. Currently, there are several approaches for trying to resolve these limitations.

Removal of hydrogen sulfide from biogas can be achieved by physical and chemical methods, and by injecting oxygen or air into the reactor headspace (Martens 2008), and by anaerobic/aerobic coupling (Guiot 1997c).

Several studies have demonstrated increased methane production under microaerobic conditions, i.e. at low dissolved oxygen concentrations (Shen 1996). The co-existence of methanogenic and aerobic microorganisms in a microbial biofilm has been demonstrated and used to develop a coupled aerobic-anaerobic biodegradation process (Guiot 1997a; Guiot 1997b; Frigon 1999; Guiot 2004; Guiot 2007; Frigon 2007). In this process oxygen and hydrogen were supplied by electrolysis of water directly in the reactor or in the external recirculation loop of the reactor and the gasses were used to achieve mineralization of chlorinated compounds in a two-step anaerobic/aerobic biodegradation process. A near-complete consumption of oxygen introduced to the reactor was observed, such that the reactor off-gas contained only small amounts of oxygen and volatilization losses of chlorinated compounds were minimized.

The insertion of electrodes in a waste holding tank (i.e. septic tank) produces the oxygen needed for the enhanced biodegradation of organic solid waste by water electrolysis (Haas 2009).

Recent advances in the development of the microbial fuel cell (MFC) and the microbial electrolysis cell (MEC) demonstrated biocatalytic properties of microorganisms at applied voltages below 1.2 V (e.g. Rozendal 2005; Rozendal 2007). Notably, in the process of microbially catalyzed electrolysis of organic materials, electrons for hydrogen production are obtained from organic materials rather than from water electrolysis.

There remains a need for efficient methods of producing methane in anaerobic bioreactors.

### SUMMARY OF THE INVENTION

There is provided a method of producing in a bioreactor a biogas rich in methane comprising: electrolyzing water in an aqueous medium at a voltage sufficient to electrolyze water without destroying microbial growth in a range of from 1.8 V to 12 V in the presence of electrochemically active anaerobic microorganisms that biocatalyze production of hydrogen gas, with a volumetric power consumption in a range of from 0.03 Wh/L_{R} to 0.3 Wh/L_{R} and a current density of 0.01 A/cm_{E}² or lower; and, contacting a species of hydrogenotrophic methanogenic microorganisms with the hydrogen gas and carbon dioxide to produce methane.

Advantageously, water electrolysis in the presence of electrochemically active microorganisms results in improved electrolysis efficiency while avoiding the use of noble metal catalysts. Further, a combination of water electrolysis with anaerobic degradation of organic matter results in increased biogas quality and in increased biogas quantity. Oxidation of hydrogen sulfide by oxygen produced in water electrolysis and reduction of carbon dioxide into methane by hydrogen produced in water electrolysis contribute to the increased quality, while an increase in the rate of organic matter hydrolysis and an increase in the production of methane from hydrogen contributes to the increased quantity.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 depicts three embodiments of an anaerobic bioreactor for implementing a method of the present invention in which: A—water electrolysis takes place within the reactor, B—water electrolysis takes place within an external recirculation loop, or C—water electrolysis takes place within an external bio-electrolyzer or electrolyzer;

FIG. 2 depicts an embodiment of an anaerobic bioreactor for implementing a method of the present invention depicting means for controlling oxygen concentration in biogas produced in the bioreactor; and,

FIG. 3 depicts a graph comparing methane production in an anaerobic bioreactor (R-1) implementing a method of the present invention to methane production in a conventional anaerobic bioreactor (R-0) of similar design but not implementing a method of the present invention.

### DESCRIPTION OF PREFERRED EMBODIMENTS

A theoretical voltage of at least 1.2 volts is required for water electrolysis. However, in practice, at least 1.8 volts is required to achieve water electrolysis. In the present method, a minimum voltage of 1.8 volts, preferably a minimum of 2 volts, is applied to electrolyze water. Since the electrolysis of water is biocatalyzed by electrochemically active microorganisms, the voltage should not be so high that microorganisms are destroyed or microbial activity is inhibited. Further, the voltage is preferably not so high as to degrade other organic matter present in the water, unlike in methods in which high voltage/current density electrolysis is used in wastewater treatment. In practice, a maximum voltage of 12 volts is applied. In a preferred embodiment, a voltage in a range of from 2 volts to 6 volts is applied.

Current density for water electrolysis depends on the type of electrodes used. A current density of 0.01 A/cm_{E}² or lower is used, where cm_{E}² is surface area of the electrode. The current density is preferably in a range of from 0.001 A/cm_{E}² to 0.005 A/cm_{E}². It is an advantage of the present method that current densities may be lower than are typically used for the given electrodes in water electrolysis.

Biocatalysis of water electrolysis advantageously reduces the amount of power required for efficient electrolysis. Volumetric power consumption is in a range of from 0.03 Wh/L_{R} to 0.3 Wh/L_{R}, where R is reactor volume, particularly as the current density is 0.01 A/cm_{E}² or lower.

In order to achieve water electrolysis, any suitable method of electrolyzing water may be used. In one embodiment, electrolysis may be achieved using a pair of spaced apart electrodes, or several electrode pairs (e.g. a stack of electrodes where cathodes and anodes are placed in sequence). One electrode is a cathode at which hydrogen is formed and the other is an anode at which oxygen is formed. It is an advantage of the present invention that electrodes may comprise inexpensive, non-corrosive materials while maintaining excellent electrolysis efficiency. Thus, the use of noble metal electrodes, such as platinum electrodes, may be avoided while maintaining excellent electrolysis efficiency. Electrodes for water electrolysis are generally known in the art and preferably comprise non-noble catalytic materials, for example, stainless steel, graphite, graphite-based materials, nickel, steel, a metal alloy or a metal oxide (e.g. titanium and/or iridium oxide). Stainless steel and graphite are particularly preferred.

The electrodes preferably have sufficient surface area to sustain microbial growth and to provide the desired current density. Electrochemically active microorganisms growing on the surfaces of the electrodes reduce the amount of gas and electron exchange that must occur through liquid medium. This provides greater electrolytic efficiency. The surface area of an electrode is sufficient to sustain a current density of 0.01 A/cm_{E}² or lower, and is preferably in a range of from 10 cm² to 100 cm² per litre of reactor volume.

In addition to electrochemically active anaerobic microorganisms that biocatalyze production of hydrogen gas at the cathode, the method also preferably employs electrochemically active aerobic microorganisms for biocatalyzing production of oxygen at the anode. Electrochemically active anaerobic microorganisms include, for example, Shewanella species, Geobacter species, or mixtures thereof. Electrochemically active aerobic microorganisms include, for example, α-Proteobacteria and β-Proteobacteria, or mixtures thereof (Logan 2006).

Hydrogen produced by water electrolysis is either released to the gas phase to become a component of the biogas, or is consumed by the hydrogenotrophic methanogenic microorganisms resulting in methane production according to the following stoichiometric reaction:

4H₂+CO₂→CH₄+2H₂O

Any suitable hydrogenotrophic methanogenic microorganisms may be used to convert the hydrogen produced from water electrolysis into methane. Such hydrogenotrophic methanogenic microorganisms include, for example, Methanobacterium spp, Methanobrevibacter spp, Methanosarcina spp, Methanococcus spp. or mixtures thereof.

Carbon dioxide used by the hydrogenotrophic methanogenic microorganisms may be provided in any suitable manner, however it is an advantage of the present process that the carbon dioxide may be provided by other anaerobic microorganisms (e.g. fermentative microorganisms, acetoclastic methanogenic microorganisms, acetogenic microorganisms) which digest organic substrates in an anaerobic bioreactor. The present process results in the partial consumption of carbon dioxide produced by such other anaerobic microorganisms thereby reducing the amount of carbon dioxide released in the biogas. The release of electrolytically produced hydrogen to the biogas also advantageously improves the combustion properties of the biogas.

In a further embodiment of the method, the biogas may also be enriched with methane by digesting organic matter with fermentative microorganisms (anaerobic and/or facultative) to produce intermediate compounds, including acetate and hydrogen, and then converting acetate to methane with a second species of methanogenic microorganism. The second species of methanogenic microorganisms is capable of converting acetate to methane. The second species of methanogenic microorganisms includes, for example, Methanosaeta spp., Methanosarcina spp. or mixtures thereof. The fermentative microorganisms include, for example, Clostridium spp., Selenomonas spp., Acetobacterium spp., Pelobacter spp., Butyribacterium spp., Eubacterium spp., Lactobacillus spp., Ruminococus spp., Streptococcus spp., Propionibacterium spp., Butyrivibrio spp., Acetivibrio spp., or mixtures thereof.

Organic matter may be any material that contains matter having carbon-carbon bonds. In a preferred embodiment, the organic matter comprises waste organic medium, for example, organic solid waste, residual biomass, biosolids or sludge, or wastewater. In a preferred embodiment, the organic matter is a component of the aqueous medium in which the water electrolysis is occurring, such as in anaerobic bioreactors. In an anaerobic bioreactor, the second species of methanogenic microorganism is responsible for 60-90% of the methane production, with water electrolysis and the hydrogenotrophic methanogenic microorganisms responsible for an additional 10-40% enhancement of methane production.

Advantageously, oxygen produced by the electrolysis of water improves the rate of hydrolysis of organic matter by facultative microorganisms being used for digestion of the organic matter in an anaerobic bioreactor. Furthermore, oxygen reacts with hydrogen sulfide (H₂S), thereby decreasing the H₂S concentration in the biogas, resulting in the chemical/biological transformation of H₂S to sulfur or sulfate.

In a preferred embodiment, it is desirable to reduce oxygen release in the biogas. Oxygen concentration in the biogas may be reduced by balancing applied power with the rate of oxygen consumption. Oxygen is consumed by biological and chemical reactions (e.g. hydrolysis and degradation of organic matter, oxidation).

### Example 1

### Bioreactor Design

Bioreactors for implementing a method of the present invention may be configured in a number of suitable ways.

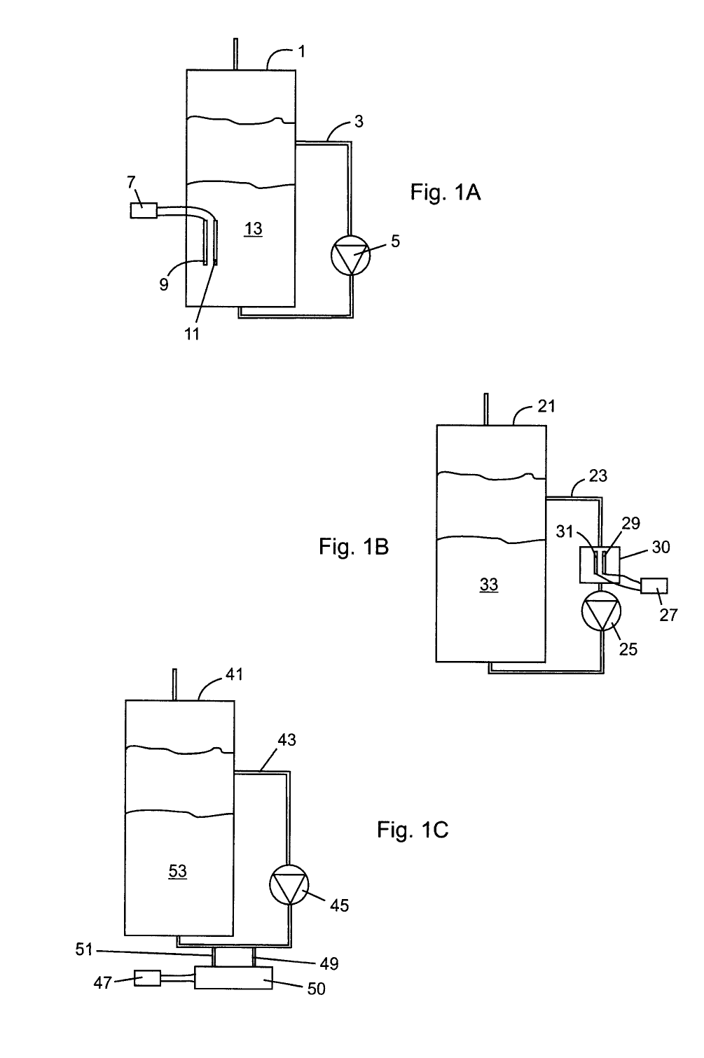

Referring to FIG. 1A, a first, and more preferred, embodiment of an anaerobic bioreactor for implementing a method of the present invention comprises a reaction vessel 1 containing sludge bed 13 composed of water, biodegradable organic materials, fermentative microorganisms for degrading organic materials, electrochemically active anaerobic and aerobic microorganisms and at least two species of methanogenic microorganisms, one species of hydrogenotrophic methanogenic microorganisms for producing methane from the hydrogen produced during electrolysis and fermentation of the organic materials and at least one other species of methanogenic microorganism (acetoclastic methanogens) for producing methane through action on acetate produced by degradation of the organic materials by the fermentative microorganisms. The bioreactor may further comprise external recirculation line 3 with pump 5 for re-circulating the sludge and liquid. Electrodes 9 and 11 installed in the sludge bed and powered by power supply 7 are used to electrolyze water into oxygen and hydrogen. The electrochemically active microorganisms in the sludge biocatalyze the electrolysis of water.

Referring to FIG. 1B, a second embodiment of an anaerobic bioreactor for implementing a method of the present invention comprises a reaction vessel 21 containing sludge bed 33 composed of water, biodegradable organic materials, fermentative microorganisms for degrading organic materials, electrochemically active anaerobic and aerobic microorganisms and at least two species of methanogenic microorganisms, one species of hydrogenotrophic methanogenic microorganisms for producing methane from the hydrogen produced during electrolysis and fermentation of the organic materials and at least one other species of methanogenic microorganism (acetoclastic methanogens) for producing methane through action on acetate produced by degradation of the organic materials by the fermentative microorganisms. The bioreactor further comprises external recirculation line 23 with pump 25 for re-circulating the sludge and liquid. Electrodes 29 and 31, located in electrolysis cartridge 30 installed in the external recirculation line, are powered by power supply 7 to electrolyze water into oxygen and hydrogen. The electrochemically active microorganisms in the sludge being re-circulated biocatalyze the electrolysis of water.

Referring to FIG. 1C, a third embodiment of an anaerobic bioreactor for implementing a method of the present invention comprises a reaction vessel 41 containing sludge bed 53 composed of water, biodegradable organic materials, fermentative microorganisms for degrading organic materials and at least two species of methanogenic microorganisms, one species of hydrogenotrophic methanogenic microorganisms for producing methane from the hydrogen produced during electrolysis and fermentation of the organic materials and at least one other species of methanogenic microorganism (acetoclastic methanogens) for producing methane through action on acetate produced by degradation of the organic materials by the fermentative microorganisms. The bioreactor further comprises external recirculation line 43 with pump 45 for re-circulating the slurry and/or liquid. An on-site bio-electrolyzer or electrolyzer 50 is used to generate oxygen and hydrogen gas by microbially catalyzed water electrolysis using electrochemically active anaerobic and aerobic microorganisms, and the hydrogen and oxygen are injected into the reactor using gas eductors 49 and 51 or any other means of gas injection into liquid.

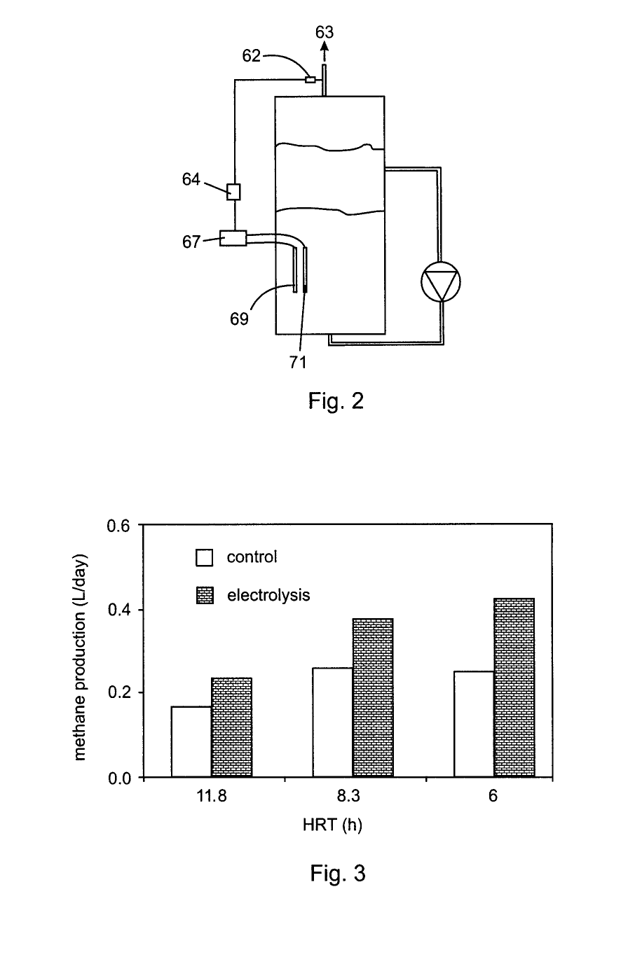

Referring to FIG. 2, power applied to the electrodes may be controlled in order to avoid or reduce accumulation of oxygen in the biogas. This can be accomplished by a feedback control system, which comprises on-line oxygen probe 62 to measure oxygen concentration in the biogas in biogas line 63, controller 64, and controllable power supply 67, which is the same power supply that supplies power to electrodes 69 and 71.

### Example 2

### Methane Production

Experiments were carried out in two 0.5 L reactors (R-0 and R-1) and in a 3.5 L UASB reactor (R-2). All reactors were inoculated with anaerobic sludge (Rougemont, Quebec, Canada). R-0 was operated as a conventional anaerobic reactor. Each test reactor (R-1 and R-2) was equipped with a pair of electrodes (stainless steel #316 cathode and titanium/iridium oxide anode) located in the sludge bed (R-1) or in the external recirculation line (R-2).

R-0 and R-1 were operated at a hydraulic retention time (HRT) of 6 h to 12 h and fed with a synthetic wastewater at an influent concentration of 650 mg/L (low strength wastewater). R-2 was operated at an HRT of 9 h and fed with synthetic wastewater at an influent concentration of 6 g/L (high strength wastewater). A power of 0.26 and 0.18 Wh/L_{R} was used in R-1 and R-2 for water electrolysis, respectively.

FIG. 3 shows a comparison of methane production in R-0 (control) and R-1 (test) reactors at different HRTs. The results show that due to water electrolysis methane production was increased by 40% or more in R-1 compared to R-0. Because of high organic load and therefore high rate of methane production in anaerobic mode, in R-2 methane production was increased by only 10-15% when compared to reactor operation without electrolysis. However, hydrogen sulfide concentration in off-gas decreased from 0.2% (anaerobic mode) to 0.01% (electrolysis mode). Also, electrolysis helped to stabilize reactor performance at a high organic load, i.e. reactor failure was avoided.

### REFERENCES

### The Contents of the Entirety of Each of which are Incorporated by this Reference

Call D F, Logan B E. High efficiency Hydrogen Gas Production Using a Microbial Electrolysis Cell (MEC).
Chapman C, Ovens D, Deegan D, Ismail S. (2007) Waste Treatment Process and Apparatus. International Patent Publication WO 2007/000607 published Jan. 4, 2007.
Chapman C, Ovens D, Deegan D, Ismail S. (2008) Waste Treatment Process and Apparatus. United States Patent Publication 2008/0097137 published Apr. 24, 2008.
Cheng S, Xing D, Call D F, Logan B E. (2009) Direct Biological Conversion of Electrical Current into Methane by Electromethanogenesis. Environ. Sci. Technol. Mar. 26, 2009.
Chenlin L, Fang H H P. (2007) Fermentative hydrogen production from wastewater and solid wastes by mixed cultures. Critical Reviews in Environmental Science and Technology. 32, 1-39.
Connelly R W, Smith D. (2007) High Performance Sewer System. International Patent Publication WO 2007/036027 published Apr. 5, 2007.
Diz H R, Felder M S, Felder J. (2006) Method of Producing Hydrogen Gas in a Bioreactor with Substrates and Associated Apparatus. United States Patent Publication 2006/0281163 published Dec. 14, 2006.
Frigon J C, Stephenson R J, Larabée S, Guiot S R. (1999) Biotreatment of resin acids by a coupled anaerobic/aerobic integrated system. Pulp & Paper Canada. 100(5):T131-T134.
Frigon J C, Bruneau T, Moletta R, Guiot S R. (2007) Coupled anaerobic-aerobic treatment of whey wastewater in a sequencing batch reactor: proof of concept. Water Science and Technology. 55(10):209-216.
Guiot S R. (1997a) Anaerobic and aerobic integrated system for biotreatment of toxic wastes (CANOXIS). U.S. Pat. No. 5,599,451 issued Feb. 4, 1997.
Guiot S R. (1997b) Process coupling of anaerobic and aerobic biofilms for treatment of contaminated waste liquids. In: D. L. Wise (Ed.), Global Environmental Biotechnology: Proc. Int. Symp. (3rd: 1996: Boston, Mass. USA) of the Int. Society for Environ. Biotech. (Studies in Environmental Sciences vol. 66), Elsevier Science, Amsterdam, pp. 591-601 (ISBN 0-444-82534-7).
Guiot S R, Darrah B, Hawari J A. (1997c) Hydrogen sulfide removal by anaerobic/aerobic coupling. Proc. 8th International Conf. on Anaerobic Digestion. May 25-29, Sendai, Japan (T. Noike, Ed.) Vol. 2, pp. 64-71.
Guiot S R, Tartakovsky B. (2005) Bioelectrolytical methanogenic/methanotrophic coupling for bioremediation of ground water. International Patent Publication WO 2005/115930 published Dec. 8, 2005.
Guiot S R, Tartakovsky B. (2007) Bioelectrolytical methanogenic/methanotrophic coupling for bioremediation of ground water. United States Patent Publication 2007/0218540 published Sep. 20, 2007.
Haas J. (2009) Biogas Capture and/or Collection System. International Patent Publication WO 2009/043141, published Apr. 9, 2009.
Jiang L, Jones T G J, Mullins O C, Wu X. (2001) Hydrogen Sulphide Detection Method and Apparatus. International Patent Publication WO 2001/063094 published Aug. 30, 2001.
Lee H-S, Torres C I, Parameswaran P, Rittmann B E. (2009) Fate of H in an Upflow Single-Chamber Microbial Electrolysis Cell Using a Metal-Catalyst-Free Cathode. Environ. Sci. Technol. Apr. 9, 2009, DOI: 10.1021/es900204.
Leite J A C, Fernanded B S, Pozzi E, Chinalia F A, Maintinguer S I, Varesche M B A, Foresti E, Pasotto M B, Zaiat M. (2006) Application of an anaerobic packed-bed bioreactor for the production of hydrogen and organic acids. World Hydrogen Energy Conference (WHEC 16). Jun. 13-16, 2006.
Lizama H M, Scott T C, Scott C D. (1996) Apparatus and Method for the Desulfurization of Petroleum by Bacteria. International Patent Publication WO 96/12564 published May 2, 1996.
Logan B E, Regan J M. (2006) Electricity-producing bacterial communities in microbial fuel cells. Trends in Microbiology. 14: 512-518.
Logan B E, Call D, Cheng S, Hamelers H V M, Sleutels T H J A, Jeremiasse A W, Rozendal R A. (2008) Microbial Electrolysis Cells for High Yield Hydrogen Gas Production from Organic Matter. Environ. Sci. Technol. 42(23): 8630-8640.
Martens C. (2008) Process for Decomposing Hydrogen Sulfide by Means of Feeding Oxygen. United States Patent Publication 2008/0227081 published Sep. 18, 2008.
National Research Council of Canada (NRC) Technology Offering L-11390. (2007) Versatile water electrolysis-based approach for integrated bioremediation of liquids contaminated with chlorinated and recalcitrant compounds.
Pitts M M, Romo R F V. (2008) Capacitive Electrostatic Process for Inhibiting the Formation of Biofilm Deposits in Membrane-Separation Systems. United States Patent Publication 2008/0156633 published Jul. 3, 2008.
Romo R F V. (2001) Electrostatic Enhancement for Membrane-Separation Systems. International Patent Publication WO 2001/026768 published Apr. 19, 2001.
Rozendal R A, Buisman C J N. (2005) Process for producing hydrogen. International Patent Publication WO 2005/005981 published Jan. 20, 2005.
Rozendal R A, Buisman C J N. (2007) Process for producing hydrogen. United States Patent Publication 2007/0042480 published Feb. 22, 2007.
Shen C F, Guiot S R. (1996) Long-term impact of dissolved O₂ on the activity of anaerobic granules. Biotechnol. Bioeng. 49(6), 611-620.
Tartakovsky B, Manuel M-F, Neburchilov V, Wang H, Guiot S R. (2008a) Hydrogen Production in a Continuous Flow Microbial Fuel Cell with a Gas Phase Cathode. Slide presentation May 29, 2008.
Tartakovsky B, Manuel M-F, Wang H, Guiot S R. (2008b) High rate membrane-less microbial electrolysis cell for continuous hydrogen production. International Journal of Hydrogen Energy. 34(2): 672-677.
Tartakovsky B, Manuel M-F, Neburchilov V, Wang H, Guiot S R. (2008c) Biocatalyzed hydrogen production in a continuous flow microbial fuel cell with a gas phase cathode. Journal of Power Sources. 182: 291-297.
Van Vliet D, Campbell H W, Chambers S B. (2004) Treatment of a Waste Stream Through Production and Utilization of Oxyhydrogen Gas. United States Patent Publication 2004/0099599 published May 27, 2004.
Van Vliet D R, Campbell H W, Chambers S B. (2008a) Treatment of a Waste Stream Through Production and Utilization of Oxyhydrogen Gas. United States Patent Publication 2008/0006584 published Jan. 10, 2008.
Van Vliet D R, Campbell H W, Chambers S B. (2008b) Removal of Contaminants from a Waste Stream Through Production and Utilization of Oxyhydrogen Gas. International Patent Publication WO 2008/064460 published Jun. 5, 2008.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of producing in a bioreactor a biogas richer in methane than before conducting the method, the method comprising:
(a) electrolyzing water using anode and cathode electrodes in an aqueous medium at a voltage sufficient to electrolyze water without destroying microbial growth in a range of from 1.8 V to 12 V in the presence of electrochemically active anaerobic microorganisms growing on the cathode that biocatalyze production of hydrogen gas, with a volumetric power consumption in a range of from 0.03 Wh/L_{R} to 0.3 Wh/L_{R} and a current density of 0.01 A/cm_{E}² or lower; and,
(b) contacting a species of hydrogenotrophic methanogenic microorganisms with the hydrogen gas and carbon dioxide to produce methane.

2. The method according to claim 1, wherein the voltage is in a range of from 2 V to 6 V.

3. The method according to claim 1, wherein the current density is in a range of from 0.001 A/cm_{E}² to 0.005 A/cm_{E}².

4. The method according to claim 1, further comprising digesting organic matter with fermentative microorganisms to produce the carbon dioxide.

5. The method according to claim 4, wherein the fermentative microorganisms further produce acetate, and the acetate is contacted with a second species of methanogenic microorganisms to produce methane.

6. The method according to claim 4, wherein the fermentative microorganisms comprise facultative microorganisms and oxygen produced during the electrolysis of water improves rate of digestion of the organic matter by the facultative microorganisms.

7. The method according to claim 4, wherein oxygen produced during the electrolysis of water reduces hydrogen sulfide concentration in the biogas.

8. The method according to claim 6, wherein applied power is balanced with rate of oxygen consumption to reduce concentration of oxygen in the biogas.

9. The method according to claim 4, wherein the organic matter is a component of the aqueous medium in which the water electrolysis is occurring.

10. The method according to claim 1, wherein electrochemically active aerobic microorganisms biocatalyze production of oxygen gas during the electrolysis of water.

11. The method according to claim 1, wherein the electrodes have sufficient surface area to provide the current density and to sustain microbial growth thereon.

12. The method according to claim 11, wherein the surface area is in a range of from 10 cm² to 100 cm² per litre of reactor volume.

13. The method according to claim 11, wherein the electrodes comprise a non-noble catalytic material.

14. The method according to claim 11, wherein the electrodes comprise stainless steel, graphite, a graphite-based material, nickel, steel, a metal alloy or a metal oxide.

15. The method according to claim 11, wherein the electrodes comprise stainless steel or graphite.

16. The method according to claim 1, wherein the electrochemically active anaerobic microorganisms comprise Shewanella species, Geobacter species, or mixtures thereof.
